(19) United States
(12) Patent Application Publication
(10) Pub. No.: US 2006/0088582 A1
(43) Pub. Date: Apr. 27, 2006

(54) Phosphatidyl oligo glycerins and structural analogs

(75) Inventor: Eibl Hansjorg, Bovenden (DE)

Correspondence Address: ROTHWELL, FIGG, ERNST & MANBECK, P.C., 1425 K STREET, N.W., SUITE 800, WASHINGTON, DC, 20005, US

(73) Assignee: Max-Planck-Gesellschaft zur Foerderung der Wissenschaften e.V., Muenchen (DE)

(21) Appl. No.: 10/514,162

(22) PCT Filed: May 12, 2003
(86) PCT No.: PCT/EP03/04942

§ 371 Date: Nov. 12, 2004

(30) Foreign Application Priority Data
May 13, 2002 (DE) 102 21 183.3

(51) Int. Cl. A61K 9/127; C07F 9/02

(52) U.S. Cl. 424/450; 558/177

(57) ABSTRACT

This invention relates to novel structural analogs to phosphatidyl oligo glycerins. These compounds can be used, in particular, for producing liposomes having a long circulation time with or without thermolability. The invention also relates to liposomes containing compounds of the aforementioned type and to medicament compositions.

The invention relates to novel structural analogs of phosphatidyloligoglycerols. These compounds can be employed in particular for producing liposomes with a long circulation time with or without thermolability. The invention further relates to liposomes comprising such compounds, and medicament compositions.

Conventional liposomes usually show a residence time in the serum of up to 5 hours. However, especially when liposomes are used as carriers of active pharmaceutical ingredients, it is desirable for the residence time of liposomes in the bloodstream to be as long as possible. To prolong the life of liposomes, the so-called “stealth liposomes” were developed and have a structure based on phosphatidyl compounds which comprise an extended polyethylene glycol residue. However, stealth liposomes are high molecular weight compounds which comprise poorly defined polyethylene glycol residues. They are not accurately defined compounds because the polyethylene glycol residues have different chain lengths.

It was therefore an object of the present invention to provide compounds which can be used in particular for producing improved liposomes.

This object is achieved according to the invention by a compound of the general formula (I) 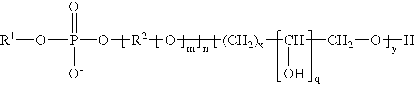
in which R¹ is a saturated or unsaturated acyl or alkyl, alkenyl or alkynyl radical which may optionally be branched or/and substituted, or is 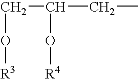
in which R³ and R⁴ are each independently of one another hydrogen, a saturated or unsaturated acyl or alkyl, alkenyl or alkynyl radical which may optionally be 10 branched or/and substituted,

R²=—(CH₂)_{z}—,

m=0 or 1,

n=an integer from 1 to 20,

q=an integer from 1 to 5,

x=an integer from 0 to 22,

y=an integer from 1 to 20 and

z an integer from 1 to 22

with the proviso that if m=0 the total of x+z≧2.

The structural elements used in the substances described herein can be varied as desired and thus tailored to the particular use.

The novel structural analogs preferably comprise in the polar region triols with terminal diols, oligoethylene-glycoglycerols or oligopropylene-glycoglycerols which can be prepared in highly pure and defined form. It is possible with these compounds to produce very different liposomes which may be stable in the serum even without addition of cholesterol. If thermolability is desired, it is in fact advantageous to work substantially without cholesterol. It is possible with the compounds of the invention in particular to produce liposomes which can be adapted exactly to the particular active ingredient employed and the therapeutic aim.

The radical R¹ in the compounds of the invention may be a saturated or unsaturated acyl or alkyl, alkenyl or alkynyl radical. This radical may optionally be branched or/and substituted, in particular by substituents selected from hydroxy, halogen, alkoxy (especially C₁-C₈-alkoxy) or other substituents. Acyl or alkyl derivatives are preferred. In a further embodiment, R¹ is a glycerol residue substituted by R³ and R⁴ in which R³ and R⁴ are each independently of one another hydrogen or a saturated or an unsaturated acyl or alkyl, alkenyl or alkynyl radical which may optionally be branched or/and substituted. Suitable substituents are, for example, hydroxy, halogen or alkoxy (especially C₁-C₈-alkoxy). If R³ and R⁴ are acyl radicals the compounds are esters, and if R³ or/and R⁴ are alkyl, alkenyl or alkynyl radicals the compounds are ethers. The invention includes diesters, monoesters, diethers, monoethers and mixed ether/ester compounds. The radicals R¹, R³ and R⁴ may include from 1 to 48 C atoms. The individual radicals include in particular from 1 to 24 C atoms, more preferably 1 to 22 C atoms. Short-chain radicals, for example with C₁-C₈, may be preferred for some applications, whereas longer-chain radicals, for example with C₁₆-C₂₂, are advantageous for other applications.

The radical R² is —(CH₂)_{z}— according to the invention. In this connection, z may be an integer from 1 to 22 and is preferably an integer from 1 to 8, in particular 2 or 3.

If m=b 0, the resulting compounds have alkyl chains, and if m=1 the compounds of the invention are glycols, for example ethylene glycols or propylene glycols.

An essential feature of the compounds of the invention is that they have a terminal diol. The compounds may, however, also have up to 6 hydroxy groups in the polar region.

x may be an integer from 0 to 22. In one embodiment, x is preferably 1 to 8, in which case the compounds are formed from triols with terminal compounds. In a further embodiment, x is preferably 1, in which case a group derived from glycerol is present in the molecule.

In a particularly preferred embodiment, the polar portion of the compounds of the invention comprises triols with terminal diol. The compounds have the formula (II): 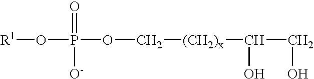
in which z 1,

m=0,

n−1,

x=an integer from 1 to 22, in particular from 1 to 8,

q=1 and

y=1.

In a further preferred embodiment, the compounds of the invention comprise in the polar region oligoethylene-glycoglycerols and have the formula (III) 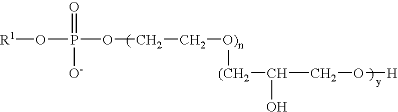
n which

z=2,

m=1,

n=an integer from 1 to 20, in particular from 1 to 4,

x=1

q=1,

y=an integer from 1 to 20, in particular from 1 to 4.

In a further preferred embodiment, the compounds of the invention comprise in the polar portion oligopropylene-glycoglycerols and have the formula (IV) 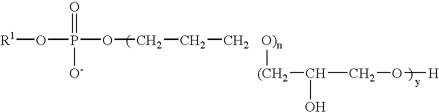
in which

z=3,

m=1,

n=an integer from 1 to 20, in particular from 1 to 3,

q=1

x=1 and

y=an integer from 1 to 20, in particular an integer from 1 to 4.

In addition, preferred compounds comprise mixed ethylene oxide and propylene oxide groups, in which therefore z is 2 at some positions and 3 at other positions.

The compounds of the invention can be obtained with exactly defined hydrophilic radicals so that the compounds can be obtained in particular as uniform compound of defined structure. The compounds are preferably >90%, more preferably >95%, particularly preferably >99% and even more preferably >99.9% uniform in relation to the value of y or/and the value of n. >90% uniform means that more than 90% of the obtained compounds have the desired chain length, i.e. the content of derivatives with a different chain length is ≦10%.

The compounds of the invention can be employed in particular in liposomes, polymerizable liposomes, lipit-containing micelles, polymerizable micelles or nanoparticles, in particular solid nanoparticles, including polymeric compositions.

The present invention therefore further relates to liposomes, micelles or nanoparticles which comprise at least one compound as described above. The liposomes, micelles and nanoparticles can be produced in a conventional way and comprise in particular at least 1, more preferably at least 10 and up to 100, more preferably up to 70, mol % of compounds of the invention. The liposomes may comprise further liposome constituents, for example phospholipids or/and alkylphospholipids. The liposomes may further comprise cholesterol, for example from 0 to 70 mol % cholesterol. However, it is also possible to produce cholesterol-free liposomes which comprise ≦1 mol % cholesterol, in particular ≦0.1 mol % cholesterol. The liposomes may advantageously further comprise one or more active pharmaceutical ingredients.

The invention further relates to liposomes which are >15% by weight formed from phosphatidyloligoglycerols or/and ≧1% by weight formed from compounds of the invention. These liposomes, which are in particular thermolabile liposomes with controlled release temperature, may advantageously also comprise a phosphatidylcholine with a main transition temperature in the range from 0 to 80° C. Phosphatidyloligoglycerols and their preparation are disclosed in DE 196 22 224.

Dipalmitoyl-sn-glycero-3-phosphodiglycerol or/and 1,2-distearoyl-sn-glycero-3-phosphodiglycerol is preferably employed. Suitable phosphatidylcholines are preferably selected from the group of 1-palmitoyl-2-olioylglycero-3-phosphocholine, 1-stearoyl-2-olioyl-3-phosphocholine, 1-palmitoyl-2-lauroylglycero-3-phosphocholine, 1-behenoyl-2-olioylglycero-3-phosphocholine, 1-stearoyl-2-lauroylglycero-3-phosphocholine, 1,3-dimyristoylglycero-2-phosphocholine, 1,2-dimyristoylglycero-3-phosphocholine, 1-palmitoyl-2-myristoylglycero-3-phosphocholine, 1-stearoyl-2-myristoylglycero-3-phosphocholine, 1-myristoyl-2-palmitoylglycero-3-phosphocholine, 1,3-palmitoylglycero-2-phosphocholine, 1,2-dipalmitoylglycero-3-phosphocholine, 1-myristoyl-2-stearoylglycero-3-phosphocholine, 1-stearoyl-3-myristoylglycero-2-phosphocholine, 1-stearoyl-2-palmitoylglycero-3-phosphocholine, 1-palmitoyl-2-stearoylglycero-3-phosphocholine, 1,3-distearoylglycero-2-phoshocholine, 1,2-distearoylglycero-3-phosphocholine, 1,2-diarachinoylglycero-3-phosphocholine, 1,2-dibehenoylglycero-3-phosphocholine and 1,2-dilignoceroylglycero-3-phosphocholine.

The content of phosphatidyloligoglycerol or of compound of the invention is preferably at least 20% by weight, more preferably at least 30% by weight and up to 100% by weight, more preferably up to 80% by weight, most preferably up to 60% by weight. Such liposomes can be prepared in particular free of cholesterol, in which case they comprise <1% by weight, more preferably <0.5% by weight and most preferably <0.1% by weight cholesterol. The liposomes preferably comprise at least 5% by weight and up to 95% by weightr preferably up to 90% by weight, phosphatidylcholine, at least 5% by weight, in particular more than 15% by weight and up to 95% by weight, in particular up to 60% by weight, phosphatidyloligoglycerol or/and a compound of the invention, and <0.5% by weight cholesterol.

Examples of particularly preferred liposomes are as follows:

As serum-unstable cholesterol-free liposome for example can be prepared from 1,2-dipalmitoyl-sn-glycero-3-phosphocholine in a molar proportion of from 10 to 90% and 1,2-dipalmitoyl-sn-glycero-3-phosphodiglycerol in a molar proportion of from 10 to 90%. The proportion of 1,2-dipalmitoyl-sn-glycero-3-phosphodiglycerol can also be partly or completely replaced by one or more compounds of the invention. A substantial advantage is that such liposomes can be produced free of cholesterol, which is important in particular for the thermolability.

It is possible by using longer-chain radicals to produce serum-stable cholesterol-free liposomes, for example by using 1,2-distearoyl-sn-glycero-3-phosphocholine in a molar proportion of from 20 to 95% and 1,2-distearoyl-sn-glycero-3-phosphodiglycerol or 1,2-distearoyl-sn-glycero-3-phosphotriglycerol in a molar proportion of from 5 to 60%. Such liposomes can also be produced free of cholesterol.

Serum-stable but thermolabile liposomes are particularly important. In this connection, it is particularly desired for the phase transition temperature to be about 41° C. Liposomes with particularly advantageous properties can be obtained by using 1,2-distearoyl-sn-glycero-3-phosphocholine (SS-GPC), 1,2-dipalmitoyl-sn-glycero-3-phosphocholine (PP-GPC) and 1,2-dipalmitoyl-sn-glycero-3-phosphodiglycerol (PP-GPG₂). Compounds like SS-GPC are used in order to achieve adequate stability in the serum. This makes it possible to produce cholesterol-free liposomes. SS-GPC is also suitable for shifting, in combination with PP-GPC, the phase transition temperature into the desired range on the temperature scale (see FIG. 1). Liposomes with 90 mol % PP-GPC, 10 mol % PP-GPG₂ and 0 mol % SS-GPC have a phase transition temperature (T_{τ}) in ° C. of 39.5, liposomes with 80 mol % PP-GPC, 10 mol % PP-GPG₂ and 10 mol % SS-GPC have a phase transition temperature Tₜ in ° C. of about 40.5, and liposomes with 70 mol % PP-GPC, 10 mol % PP-GPG₂ and 20 mol % SS-GPC have a phase transition temperature Tₜ in ° C. of about 41.

The release of active ingredients entrapped in liposomes, for example carboxyfluorescin, depends on the PP-GPG₂ content (see FIG. 2). Release of the entrapped material reaches >90% with a content of 30 mol % PP-GPG₂, 50 mol % PP-GPC and 20 mol % of SS-GPC.

The nanoparticles of the invention can be composed of lipids or other materials.

The compounds of the invention cause in particular a surface modification of liposomes, micelles and nanoparticles and thus increase their life or the breakdown time. The compounds of the invention can therefore be employed in particular for increasing the circulation time, for example after i.v. injection of liposomes, micelles or nanoparticles. Such systems can be employed particularly preferably for delayed release of active ingredient. It is thus possible to use pulsed administration protocols, or single doses of active ingredients in order to deliver the correct amount of an active ingredient which is required to treat the particular disorder.

The active ingredients of the invention, in particular as constituent of liposomes, micelles or nanoparticles, may be in particular a constituent of a pharmaceutical composition, where appropriate together with further conventional diluting adjuvant carriers or/and fillers.

The pharmaceutical composition may be intended in particular for parenteral or oral administration or for administration by inhalation. It is also possible in particular to achieve delayed and controlled delivery of active ingredients via the route of administration through the lungs. Suitable dosage forms for administration as inhalants include for example dry powders, particles, solid nanoparticles, liposomes, emulsions, micelles, complexes, suspensions, solutions etc. Examples suitable for parenteral administration are liposomes, emulsions, micelles, complexes, suspensions, in particular suspensions with particles or solid nanoparticles, and solutions. The compounds of the invention can be formulated for oral administration for example as capsules, tablets, in particular tablets with interic coatings, with the formulation intended for oral administration including in particular a dry powder, particles, solid nanoparticles, liposomes, emulsions, micelles, complexes, suspensions, self-emulsifying formulations or formulations with delayed release.

The following routes are particularly preferred for administration: . . . (bd), intrabronchial (br), intradermal (dl), intraarterial (ia), intragastritic (ig), inhaling (ih), intramuscular (im), intraperitoneal (ip), intravenous (iv), parenteral (pa), subcutaneous (sc), intraspinal (sp), transdermal (td), topical (tp) or intravaginal (va) administration.

The invention is explained further by the following examples and appended figures, in which

FIG. 1 shows the thermolability of liposomes depending on the proportion of SS-GPC in PP-GPG₂ (10%)/PP-GPC liposomes;

FIG. 2 shows the thermolability of liposomes depending on the proportion of PP-GPG₂ in SS-GPC (20%)/PP-GPC liposomes.

### EXAMPLES

Group A

Polar portion: triols with terminal diol

Synthesis via protected diol (isopropylidene protective group)

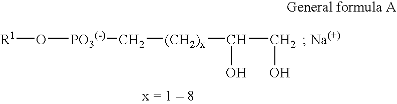

### Examples

### 1) 1,2-Dipalmitoyl-sn-glycero-3-phosphobutanetriol-(1.2.4);

| | |
|---|---|
| Na⁽⁺⁾ salt | (x = 1) |
| C₃₉H₇₆NaO₁₀P | (MW 758.991) |

| | | | | | | |
|---|---|---|---|---|---|---|
| Calc. | C | 61.72 | H | 10.09 | P | 4.08 |
| Found | | 61.48 | | 9.98 | | 4.05 |

### 2) 1,2-Distearoyl-sn-glycero-3-phosphobutanetriol-(1.2.4);

| | |
|---|---|
| Na⁽⁺⁾ salt | (x = 1) |
| C₄₃H₈₄NaO₁₀P | (MW 815.099) |

| | | | | | | |
|---|---|---|---|---|---|---|
| Calc. | C | 63.36 | H | 10.39 | P | 3.80 |
| Found | | 63.12 | | 10.28 | | 3.65 |

### 3) 1,2-Dimyristoyl-sn-glycero-3-phosphooctanetriol-(1.2.8);

| | |
|---|---|
| Na⁽⁺⁾ salt | (x = 5) |
| C₃₉H₇₆NaO₁₀P | (MW 758.991) |

| | | | | | | |
|---|---|---|---|---|---|---|
| Calc. | C | 61.72 | H | 10.09 | P | 4.80 |
| Found | | 61.24 | | 10.01 | | 4.03 |

### 4) 1-Stearoyl-2-myristoyl-sn-glycero-3-phosphobutane-triol-(1.2.4);

| | |
|---|---|
| Na⁽⁺⁾ salt | (x = 1) |
| C₃₉H₇₆NaO₁₀P | (MW 758.991) |

| | | | | | | |
|---|---|---|---|---|---|---|
| Calc. | C | 61.72 | H | 10.09 | P | 4.08 |
| Found | | 61.59 | | 10.04 | | 4.01 |

It is possible correspondingly to obtain further fatty acid combinations and corresponding monoacyl derivatives and alkyl derivatives.

Group B

Polar portion; ethylene glycoglycerols

Synthesis via protected diol (isopropylidene protective group)

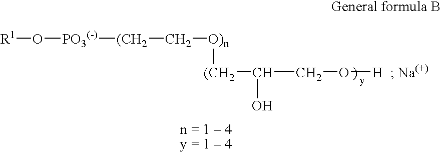

### Examples

### 5) 1,2-Dipalmitoyl-sn-glycero-3-phosphoethylene-glycoglycerol;

| | |
|---|---|
| Na(+) salt | (n = 1; y = 1) |
| C₄₀H₇₈NaO₁₁P | (MW 789.017) |

| | | | | | | |
|---|---|---|---|---|---|---|
| Calc. | C | 60.89 | H | 9.66 | P | 3.43 |
| Found | | 60.69 | | 9.51 | | 3.67 |

### 6) 1,2-Dipalmitoyl-sn-glycero-3-phosphodiethylene-glycoglycerol;

| | |
|---|---|
| Na(+) salt | (n = 2; y = 1) |
| C₄₂H₈₂NaO₁₂P | (MW 833.090) |

| | | | | | | |
|---|---|---|---|---|---|---|
| Calc. | C | 60.56 | H | 9.92 | P | 3.72 |
| Found | | 60.47 | | 9.85 | | 3.63 |

### 7) 1,2-Dipalmitoyl-sn-glycero-3-phosphoethylene-glycodiglycerol;

| | |
|---|---|
| Na(+) salt | (n = 1; y = 2) |
| C₄₃H₈₄NaO₁₃P | (MW 863.096) |

| | | | | | | |
|---|---|---|---|---|---|---|
| Calc. | C | 59.84 | H | 9.81 | P | 3.59 |
| Found | | 59.51 | | 9.79 | | 3.40 |

### 8) 1,2-Distearoyl-sn-glycero-3-phosphodiethylene-glycoglycerol;

| | |
|---|---|
| Na(+) salt | (n = 2; y = 1) |
| C₄₆H₉₀NaO₁₂P | (MW 889.188) |

| | | | | | | |
|---|---|---|---|---|---|---|
| Calc. | C | 62.14 | H | 10.20 | P | 3.48 |
| Found | | 62.10 | | 10.08 | | 3.35 |

### 9) 1,2-Distearoyl-sn-glycero-3-phosphoethylene-glycodiglycerol;

| | |
|---|---|
| Na(+) salt | (n = 1; y = 2 |
| C₄₇H₉₂NaO₁₃P | (MW 919.204) |

| | | | | | | |
|---|---|---|---|---|---|---|
| Calc. | C | 61.41 | H | 10.09 | P | 3.37 |
| Found | | 61.27 | | 9.99 | | 3.31 |

### 10) 1-Stearoyl-2-palmitoyl-sn-glycero-3-phospho-ethylene-glycodiglycerol;

| | |
|---|---|
| Na(+) salt | (n = 1; y = 2) |
| C₄₅H₈₈NaO₁₃P | (MW 891.150) |

| | | | | | | |
|---|---|---|---|---|---|---|
| Calc. | C | 60.65 | H | 9.95 | P | 3.48 |
| Found | | 60.43 | | 9.81 | | 3.31 |

### 11) Octadecylphosphoethylene-glycoglycerol;

| | |
|---|---|
| Na(+) salt | (n = 1; y = 1) |
| C₂₃H₄₈NaO₇P | (MW 490.594) |

| | | | | | | |
|---|---|---|---|---|---|---|
| Calc. | C | 56.31 | H | 9.86 | P | 6.31 |
| Found | | 56.18 | | 9.82 | | 6.29 |

### 12) Octadecylphosphodiethylene-glycoglycerol;

| | |
|---|---|
| Na(+) salt | (n = 2; y = 1) |
| C₂₅H₅₂NaO₈P | (MW 534.647) |

| | | | | | | |
|---|---|---|---|---|---|---|
| Calc. | C | 56.16 | H | 9.80 | P | 5.99 |
| Found | | 56.01 | | 9.74 | | 5.54 |

### 13) Oleylphosphodiethylene-glycoglycerol;

| | |
|---|---|
| Na(+) salt | (n = 2; y = 1) |
| C₂₅H₅₀NaO₈P | (MW 532.631) |

| | | | | | | |
|---|---|---|---|---|---|---|
| Calc. | C | 56.38 | H | 9.46 | P | 5.82 |
| Found | | 56.21 | | 9.39 | | 5.59 |

### 14) Erucylphosphodiethylene-glycoglycerol;

| | |
|---|---|
| Na(+) salt | (n = 2; y = 1) |
| C₂₉H₅₈NaO₈P | (MW 588.739) |

| | | | | | | |
|---|---|---|---|---|---|---|
| Calc. | C | 59.16 | H | 9.93 | P | 5.26 |
| Found | | 58.93 | | 9.89 | | 4.98 |

It is possible correspondingly to obtain further fatty acid combinations and corresponding monoacyl derivatives and alkyl derivatives.

Group C

Polar portion: propylene glycoglycerols

Synthesis via safeguarded diol (isopropylidene protective group)

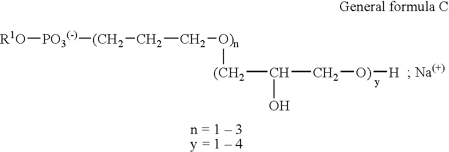

### Examples

### 15) 1,2-Dipalmitoyl-sn-glycero-3-phosphopropylene-glycoglycerol;

| | |
|---|---|
| Na⁽⁺⁾ salt | (n = 1; y = 1) |
| C₄₁H₈₀NaO₁₁P | (MW 803.044) |

| | | | | | | |
|---|---|---|---|---|---|---|
| Calc. | C | 61.32 | H | 10.04 | P | 3.86 |
| Found | | 61.18 | | 9.96 | | 3.79 |

### 16) 1,2-Dipalmitoyl-sn-glycero-3-phosphodipropylene-glycoglycerol;

| | |
|---|---|
| Na⁽⁺⁾ salt | (n = 2; y = 1) |
| C₄₄H₈₆NaO₁₂P | (MW 861.124) |

| | | | | | | |
|---|---|---|---|---|---|---|
| Calc. | C | 61.37 | H | 10.07 | P | 3.60 |
| Found | | 61.24 | | 10.01 | | 3.54 |

### 17) 1,2-Dipalmitoyl-sn-glycero-3-phosphopropylene-glycodiglycerol;

| | |
|---|---|
| Na⁽⁺⁾ salt | (n = 1; y = 2) |
| C₄₄H₈₆NaO₁₃P | (MW 877.123) |

| | | | | | | |
|---|---|---|---|---|---|---|
| Calc. | C | 60.25 | H | 9.88 | P | 3.53 |
| Found | | 60.11 | | 9.76 | | 3.39 |

### 18) 1,2-Distearoyl-sn-glycero-3-phosphodipropylene-glycoglycerol;

| | |
|---|---|
| Na⁽⁺⁾ salt | (n = 2; y = 1) |
| C₄₈H₉₄NaO₁₂P | (MW 917.232) |

| | | | | | | |
|---|---|---|---|---|---|---|
| Calc. | C | 62.86 | H | 10.33 | P | 3.38 |
| Found | | 62.14 | | 10.25 | | 3.21 |

### 19) 1,2-Distearoyl-sn-glycero-3-phosphopropylene-glycodiglycerol;

| | |
|---|---|
| Na⁽⁺⁾ salt | (n = 1; y = 2) |
| C₄₈H₉₄NaO₁₃P | (MW 933.231) |

| | | | | | | |
|---|---|---|---|---|---|---|
| Calc. | C | 61.78 | H | 10.15 | P | 3.32 |
| Found | | 61.49 | | 10.07 | | 3.19 |

### 20) Stearoyl-2-palmitoyl-sn-glycero-3-phosphopropylene-glycodiglycerol;

| | |
|---|---|
| Na⁽⁺⁾ salt | (n = 1; y = 2) |
| C₄₆H₉₀NaO₁₃P | (MW 905.177) |

| | | | | | | |
|---|---|---|---|---|---|---|
| Calc. | C | 61.04 | H | 10.02 | P | 3.42 |
| Found | | 60.89 | | 9.87 | | 3.28 |

### 21) Octadecylphosphopropylene-glycoglycerol;

| | |
|---|---|
| Na⁽⁺⁾ salt | (n = 1; y = 1) |
| C₂₄H₅₀NaO₇P | (MW 504.621) |

| | | | | | | |
|---|---|---|---|---|---|---|
| Calc. | C | 57.13 | H | 9.99 | P | 6.10 |
| Found | | 56.95 | | 9.92 | | 6.01 |

### 22) Octadecylphosphodipropylene-glycoglycerol;

| | |
|---|---|
| Na⁽⁺⁾ salt | (n = 2; y = 1) |
| C₂₇H₅₆NaO₈P | (MW 562.701) |

| | | | | | | |
|---|---|---|---|---|---|---|
| Calc. | C | 57.63 | H | 10.03 | P | 8.81 |
| Found | | 57.48 | | 9.87 | | 8.36 |

### 23) Oleylphosphopropylene-glycoglycerol;

| | |
|---|---|
| Na⁽⁺⁾ salt | (n = 1; y = 1) |
| C₂₄H₅₄NaO₇P | (MW 502.604) |

| | | | | | | |
|---|---|---|---|---|---|---|
| Calc. | C | 57.35 | H | 9.63 | P | 6.16 |
| Found | | 57.12 | | 9.47 | | 6.11 |

### 24) Oleylphosphopropylene-glycodiglycerol;

| | |
|---|---|
| Na⁽⁺⁾ salt | (n = 1; y = 2) |
| C₂₇H₅₄NaO₉P | (MW 576.684) |

| | | | | | | |
|---|---|---|---|---|---|---|
| Calc. | C | 56.24 | H | 9.44 | P | 5.37 |
| Found | | 56.17 | | 9.28 | | 5.01 |

### 25) Erucylphosphopropylene-glycodiglycerol;

| | |
|---|---|
| Na⁽⁺⁾ salt | (n = 1; y = 2) |
| C₃₁H₆₂NaO₉P | (MW 632.792) |

| | | | | | | |
|---|---|---|---|---|---|---|
| Calc. | C | 58.84 | H | 9.88 | P | 4.90 |
| Found | | 58.72 | | 9.69 | | 4.87 |

1. A compound of the general formula (I) 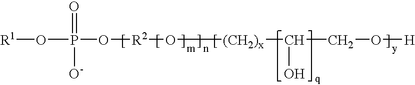
in which R¹ is a saturated or unsaturated acyl or alkyl, alkenyl or alkynyl radical which may optionally be branched or/and substituted, or is 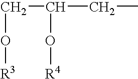
in which R³ and R⁴ are each independently of one another hydrogen, a saturated or unsaturated acyl or alkyl, alkenyl or alkynyl radical which may optionally be branched or/and substituted,
R²=—(CH₂)_{z}—,
m=0 or 1,
n=an integer from 1 to 20,
g=an integer from 1 to 5,
x=an integer from 0 to 22,
y=an integer from 1 to 20 and
z=an integer from 1 to 22
with the proviso that if m=0 the total of x+z≧2.

2. The compound as claimed in claim 1 with the formula (II) 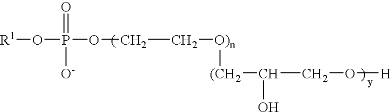
in which x is an integer from 1 to 8.

3. The compound as claimed in claim 1 with the formula (III) 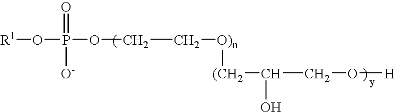
in which n is an integer from 1 to 4 and y is an integer from 1 to 4.

4. The compound as claimed in claim 1 with the formula (IV) 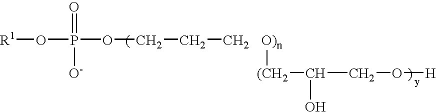
in which n is an integer from 1 to 3 and y is an integer from 1 to 4.

5. The compound as claimed in claim 1, characterized in that it is >90% uniform in relation to the value of y or/and the value of n.

6. The compound as claimed in claim 1, characterized in that m is 1 and z is 2 or 3 at every occurrence.

7. The compound as claimed in claim 1, characterized in that R¹, R³ and R⁴ each independently of one another have from 1 to 48 C atoms.

8. A liposome, characterized in that it comprises from 1 to 100 mol % of a compound as claimed in claim 1.

9. The liposome as claimed in claim 8, characterized in that it additionally comprises phospholipids or/and alkylphospholipids or/and cholesterol.

10. A liposome, characterized in that it is >15% by weight formed from phosphatidyloligoglycerol or/and ≦1% by weight formed from a compound as claimed in claim 1.

11. The liposome as claimed in claim 10, characterized in that it additionally comprises a phosphatidylcholine with a main transition temperature in the range from 0 to 80° C.

12. The liposome as claimed in claim 11, characterized in that it is a thermostable liposome with controlled release temperature.

13. A cholesterol-free liposome, characterized in that it comprises at least 5% by weight phosphatidylcholine, at least 5% by weight phosphatidyloligoglycerol or/and compounds as claimed in claim 1, and <0.5% by weight cholesterol.

14. A pharmaceutical composition comprising a compound as claimed in claim 1, together with pharmaceutically customary diluents, excipients, carriers or/and fillers.

15. A pharmaceutical composition comprising a liposome as claimed in claim 8, together with pharmaceutically customary diluents, excipients, carriers or/and fillers.
